(19) United States
(12) Patent Application Publication
(10) Pub. No.: US 2014/0243763 A1
(43) Pub. Date: Aug. 28, 2014

(54) Devices for the Placement of Medical Compounds in Natural Orifices of a Body

(71) Applicant: Sara Heikali, Los Angeles, CA (US)

(72) Inventor: Sara Heikali, Los Angeles, CA (US)

(21) Appl. No.: 14/187,651

(22) Filed: Feb. 24, 2014

Related U.S. Application Data
(60) Provisional application No. 61/850,841, filed on Feb. 25, 2013.

(51) Int. Cl. A61F 9/00

(52) U.S. Cl. CPC A61F 9/0026
USPC 604/294

(57) ABSTRACT

Methods and devices for the delivering of medicinal compounds into the natural orifice of a being, human or otherwise, but most suitably for the delivery of a punctal plug to the punctum of a human.

### CROSS-REFERENCE TO RELATED APPLICATIONS

This non-provisional patent application claims the benefit of U.S. Provisional Patent Application No. 61/850841 filed Feb. 25, 2013, entitled Rapid and Multiple Punctal Plug Inserting Instrument and Dilation, which application is incorporated in its entirety by this reference.

### BACKGROUND OF THE INVENTION

1. Technical Field

This invention is directed toward methods and apparatus for the selective and controlled placement of medicinal compounds into the natural orifices of a body be it human or otherwise. The invention discloses relatively low cost devices and methods of use that help to allow ease of medicines into natural orifices. More specifically, this invention is generally on the subject of lacrimal punctum dilation and plug insertion in the punctum of a human. The invention is related to an instrument for the dilation of the punctum and the insertion of the plug in the lacrimal punctum for the treatment of ophthalmic diseases such as dry eye disease commonly found in adult humans.

2. Background Art

Punctal plugs are presently used as a medicinal apparatus to treat dry eye disease, a disease in which the eye is characterized by dryness due to poor quality of tears, lower levels of tear production or higher levels of tear film evaporation. In this condition, the insufficiency or poor quality of tears does not allow for proper lubrication and nourishment of the eye, which is necessary for the maintenance of the health of the eye's front ocular surface, otherwise known as the cornea. Excess tears in the eye flow into small holes found on the inner corner of the upper and lower eyelid margins of the eye. These individual holes are anatomically identified and named lacrimal punctum. Through these holes, excess tears are drained into other small channels named lacrimal ducts, which drain the excess tears off into the back of the nose. When the eyelid blinks, the non-excess tears are spread and circulated across the eye's front ocular surface to provide clear vision.

Individuals who suffer from dry eye disease also called “keratoconjunctivitis sicca,” “keratitis sicca” or “dry eye syndrome” experience symptoms such as eye irritation, burning, foreign body sensation, and blurred vision. One of the treatments is to prevent the tear drainage by the occlusion or obstruction of the lacrimal punctum. Punctal occlusion can be accomplished through permanent punctal cautery, permanent ablation or temporary, extended or permanent punctal plugs. Punctal cautery is a procedure whereby the punctum is surgically sealed with the use of a heated wire tip of a cautery device inserting into the punctum, causing burning, shrinking, and scarring of the punctum. Punctal ablation is when an argon laser is focused on the punctal opening and causes for punctal ‘welding.’ Temporary, extended and permanent punctal plug occlusions, the less invasive options, are when the punctum is blocked using collagen, PCL (E-Caprolactone-L-Lactide copolymer) or silicone punctal plugs respectively. More recently, some permanent punctal plugs are made of the thermodynamic acrylic polymer or hydrogel, which conform to the space of the punctum more accurately or are more easily irrigated and removed from the punctum respectively.

The procedure of inserting a punctal plug in a punctum to achieve punctal occlusion begins with dilating the punctum and afterwards inserting the plug into the punctum. A typical collagen or PCL punctal plug is cylindrical. The typical range of sizes for temporary or extended punctal plugs is 0.2 mm-0.7 mm in diameter with a typical length of 2.0 mm. A typical silicone punctal plug is composed of a cylindrical base with a conical head with a coaxial hole at the end of the cylindrical portion. The coaxial hole at the end of the cylindrical portion of the silicone punctal plug is for the purpose of the punctal plug sitting on the tip of an insertion instrument for stable support on the instrument. A punctum plug size must properly fit the size of the subject's punctum and must be inserted at the optimum depth in relation to the surface of the punctum. If the punctum plug is too loose, the punctal plug may fall out of the punctum. If the punctum plug is too tight, the punctal plug may cause punctal stretching and discomfort to the subject. Therefore, the precise insertion f the punctum plugs into the punctum may determine and influence the level of punctal plug effectiveness in treating signs and symptoms of dry eye disease.

An assortment of instruments is known for inserting permanent punctal plugs into the punctum. However, very few instruments are known for inserting temporary and/or extended punctal plugs into the punctum. The instrument most commonly used for the insertion of temporary or extended punctal plugs is the jeweler's forceps. The jeweler's forceps has slender flat legs at the head with a narrowed, fine pointed, smooth tipped blade.

The jeweler's forceps is a microvascular instrument intended to assist in performing procedures on small vessels and determining the patency of vessels. More specifically, it can be used as a needle holder aiding with placement of sutures in a fine microvascular anastomosis, a cross connection constructed surgically between neighboring blood vessels. A jeweler's forceps is reputably used for grasping and handling tissues in microsurgical procedures.

In ophthalmic/optometric procedures, a jeweler's forceps is utilized to remove loose foreign body or sutures in or around the eye, epilate lashes, and insert punctal plugs in the punctum. Currently, temporary and extended punt al plugs are typically packaged, arranged and preloaded into a piece of foam. Utilization of punctal plugs would require initial careful precise removal of the plug from the foam in an upright vertical position. Placing thumb pressure on the flat legs location near the blades of the jeweler's forceps allows for the gripping of the punctal plug. However, the plug must be withdrawn from the foam in an upright vertical position and cannot be tilted or canted to one side or another; since the blades of the jeweler's forceps are not shaped to embrace the entire circumference of the plug, the plug can easily be canted to one side or another. Once one end of the upright, vertically positioned, cylindrical punctal plug is placed within the punctum, releasing thumb pressure would release the plug into the punctum.

Maintaining the upright vertical position of the plug during attempts to place the plug in the punctum is difficult, as such precision is difficult to achieve. Also, initial thumb pressure on the flat legs of the forceps to clasp the plug must not be in excess, otherwise it may distort the matrix integrity of the punctal plug and distort its shape. The initial appropriate amount of thumb pressure must not be altered during clasping of the punctal plug, since it may either distort and deform the plug or release the plug untimely. In addition, once the plug is dislodged from the jeweler's forceps, given it is placed in the vertical direction of the punctum, the optimum depth placement of the plug is difficult to measure. In addition, using the jeweler's forceps during a punctal plug insertion procedure requires subtle manipulation and balance of the punctal plug between the jeweler's forceps blades. Moreover the manipulation of the punctal plug and insertion of the punctal plug must be completed within five seconds of dilating the punctum, as the punctal hole shrinks within approximately five seconds after dilation.

During the punctal plug insertion procedure, the jeweler's forceps is brought close to the punctum, since the jeweler's forceps is very pointed in its end, it could be potentially dangerous to be working with the jeweler's forceps near the eye. The jeweler's forceps is typically made of stainless steel. Though under a pound in weight, if held by the arm raised in front of the eye for over a couple minutes in manipulation, efforts may become tiresome. In addition, efforts to manipulate the punctum plug properly and insert the plug in the punctum, the plug may come in contact with unwanted materials which may not be sterile; if the plug touches a non-sterile item, it will be contaminated and if inserted in the punctum it may cause complications, such as canaliculitis, dacrocystitis or other infections of the lacrimal system.

Typically punctal plug companies sell and promote two types of jeweler's forceps: straight and curved. Straight denotes a jeweler's forceps that has slender flat legs at the head continuing straight with a narrowed, fine pointed, smooth tipped blade. Curved denotes a jeweler's forceps that has slender flat legs at the head continued in curved manner with a narrowed, fine pointed, smooth tipped blade. The curved jeweler's forceps gives a benefit over the straight jeweler's forceps in that it shifts the flat legs and the doctor's fingers out of the doctor's field of view. All other problems remain the same.

One company sells a temporary and/or extended punctal plug insertion instrument called a grooved forceps that has slender flat legs at the head with a swan-necked, angled and grooved terminal end. In ophthalmic procedures, the grooved forceps are used in cataract section suturing and corneal graft surgeries. That company sells and promotes these grooved forceps as an instrument for temporary and extended punctal plug insertion. The swan neck shifts the flat legs and the doctor's fingers out of the doctor's field of view. The grooved ends which are 0.5 mm broad can clasp punctal plugs of 0.5 mm diameters, which correspond to the typical punctal plug diameter size of most subjects. However, this 0.5 mm broad grooved end is not well suited for other punctal plugs sizes with other diameters. The grooved end allows for the grasping of the punctal plug without making an accidental puncture to the surrounding region of the punctum or the eye itself. The grooved ends with thumb pressure on the flat legs corresponds to the cylindrical shape of the punctal plug during clasping of the plug; however, the grooved forceps has no mechanical mechanism for inserting the punctal plug in an upright vertical position into the punctum at its optimum depth to the surface of the punctum.

Moreover, having two different separate instruments for dilating the punctum and inserting the plug into the punctum requires handling more devices and slows down the punctal plug insertion procedure. In addition, if some steps in the procedure are not done swiftly enough, some procedures may need to be repeated. Clasping a punctal plug by current convention requires both more time and finesse for punctal plug handling especially because they are handled one by one. It is desirable to have one instrument that functions to both dilate and insert multiple punctal plugs that can additionally store multiple sterile punctal plugs and release sterile punctal plugs into the punctum in an upright vertical position in an optimum depth to the punctum with easy manipulation of the punctum plugs and good instrument ergonomics. A light weight instrument that can incorporate all these features can expedite the punctal plug dilation and insertion procedure in multiple punctum in one subject in a more efficient manner with less manipulative difficulty.

### BRIEF SUMMARY OF THE INVENTION

It is therefore an object of the invention to provide a punctum dilating and punctum inserting instrument that has a dilator on one end of the instrument and an inserter on the other end of the instrument.

It is another object of the invention to provide a punctum dilating and punctum inserting instrument that can be preloaded with multiple sterile punctal plugs of a specific diameter for insertion on one end and a corresponding diameter sized dilator on the other end.

It is another object of the invention to provide a punctum dilating and/or punctum inserting instrument that can maintain preloaded sterile punctal plugs in a single instrument and thus allows for multiple dilations of punctum and insertions of punctal plugs in a patient without need for intermission during punctal dilation and plug inserting procedure for opening and handling of plugs such as is needed when multiple instruments are utilized, as when there is a need for reaching for and using a second instrument.

It is another object of the invention to provide a plug inserting instrument that dependably, easily and efficiently releases the plug into the punctum in an upright vertical position.

It is another object of the invention to provide in some instances a punctum dilating and plug inserting instrument that dependably, easily, and efficiently releases the plug into the punctum at an optimum depth in relation to the surface of the punctum.

It is still further another object of the invention to provide a medical instrument that is cost-effective in production, so that the sterile punctal plug encasement is disposable after a single use.

It is another object of the invention to provide a punctal plug inserting instrument that is capable of delivering multiple or individual punctal plugs, individually or multiple at a time.

It is another object of the invention to provide a plug inserting and in some cases a punctum dilator instrument that is capable of storing multiple sterile punctal plugs therein.

It is another object of the invention to provide a punctum dilating and punctum inserting instrument that is capable of increasing time efficiency in terms of dilating the punctum and delivering a punctal plug(s).

It is another object of the invention to provide a punctum dilating and punctum inserting instrument that is shaped and designed for the precise manipulation and handling of punctal plugs.

It is another object of the invention to provide a punctum dilating and punctum inserting instrument that reduces the risk of punctal plug droppage and excess material waste resulting from punctal plug material delivery and/or packaging waste.

It is another object of the invention to provide a punctum dilating and punctum inserting instrument that is capable of storing and delivering punctal plugs without distorting the integrity of the punctal plug matrix.

It is another object of the invention to provide a modular element that is easily adjoined or affixed to mechanical lead pencils, such that the punctal plug dilator attaches to the area in which the eraser was housed, and that the modular sterile punctal plug enclosure and dispensing tip attaches or is affixed to the dispensing side of the mechanical lead pencil, once a minor modification is made to the mechanical lead pencil for stable and straight affixation.

It is additionally an object of the invention to have a metal rod fabricated to replace the graphite ‘lead’ that is customarily dispensed from the inner housing of the mechanical lead pencil, such that the advancement of the metal rod will dispense the punctal plugs, without directly touching them, allowing for the dispensing of the punctal plug(s) without compromising their sterility.

It is another object of the invention to provide a barrier member that separates the sterile punctal plugs within the sterile housing from the metal extruding rod, such that the punctal plugs remain sterile.

It is another object of the invention to have the metal extruding rod be of an appropriate measured length and design with a stopper at the end, such that it cannot be extruded past a measured ending point within the previously utilized housing for the graphite ‘lead’ within the mechanical lead pencil's internal housing where the barrier behind the sterile punctal plugs cannot extrude past the tip of the housing as a safety measure prohibiting the accidental extrusion of the barrier.

It is still further an object of the invention to have a medical compound dispensing device having a radially spaced actuator for discreet dispensing of medicaments and wherein instruments of the invention may be fabricated for sterile one time use.

Further objects and embodiments of the invention will become apparent as the description proceeds herein.

### BRIEF DESCRIPTION OF THE DRAWINGS

FIG. 1 is a schematic illustration of a human eye illustrating one punctum or tear duct on the lower eyelid and another on the upper eyelid;

FIG. 2 is an exploded view of one embodiment of the invention, illustrating components thereof;

FIG. 3 is a cross sectioned view illustrating one embodiment of the invention using a conventional side actuated mechanical lead pencil;

FIG. 4 is a cross sectioned view depicting one or more embodiments of the invention utilizing in one instance the device depicted in FIG. 3; and

FIG. 5 is an enlarged cross sectioned view of the modular component of the invention,

### DETAILED DESCRIPTION OF THE INVENTION

The detailed description set forth below in connection with the appended drawings is intended as a description of presently-preferred embodiments of the invention and is not intended to represent the only forms in which the present invention may be constructed or utilized. The description sets forth the functions and the sequence of steps for constructing and operating the invention in connection with the illustrated embodiments. However, it is to be understood that the other applications of the method and devices of the invention will be readily apparent to those of ordinary skill in the art.

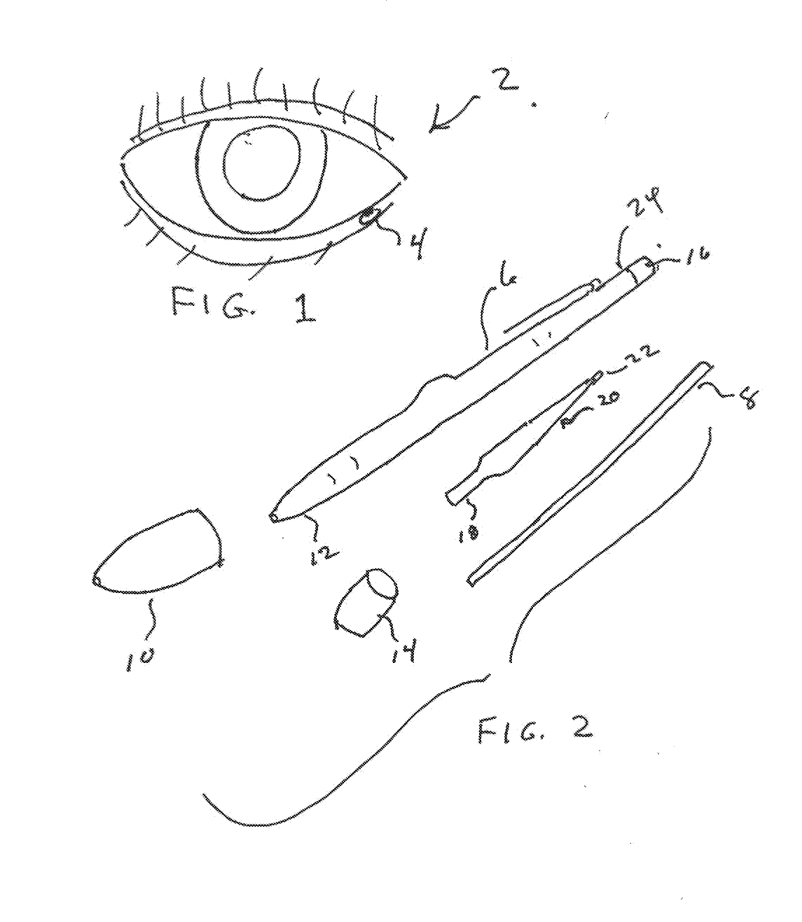

Referring to the drawings wherein like numbers of reference refer to like elements throughout, FIG. 1 depicts the schematic showing of a typical eye wherein a punctum or tear duct is shown.

FIG. 1 schematically depicts a human eye 2 having a natural orifice or tear duct 4 which is to have a punctum, not shown, inserted therein. The tear duct 4 is dilated so as to enable a medical compound or plug to be inserted into the temporarily enlarged opening.

FIG. 2 depicts, for one embodiment, the components necessary for practicing the invention and may, in some instances, comprise a kit form to enable a physician or practitioner to perform the inserting of medicaments into a natural orifice of the recipient.

There, a conventional mechanical lead pencil 6 is provided, the conventional lead removed so as to receive steel rod 8 of the same size and dimension as the removed lead, not show, for purposes as will become apparent.

The modular plug carrying member 10 is adapted to be secured to the end 12 of the mechanical pencil 6 by, in one movement having mechanical threads incised on the plastic barrel 14 of mechanical pencil 6 by means 7, thread maker 14, much like one would sharpen the end of a wooden pencil by means of a pencil sharpener. In other embodiments an electrically driven thread maker may be used. Suffice to say that internal threads of device 10 are adapted to be matingly secured to the incised threads of and 12 mechanical pencil 10.

The mechanical lead pencil 6 has eraser 16 which may be removed so as to receive end 18 of dilator 20 having tip 22 for dilating a natural orifice or in this case tear duct 4 of eye 2.

It will be noted that having the dilator 20 secured to the end 24 of mechanical pencil 6 facilities the medicament placement procedure, as will become apparent.

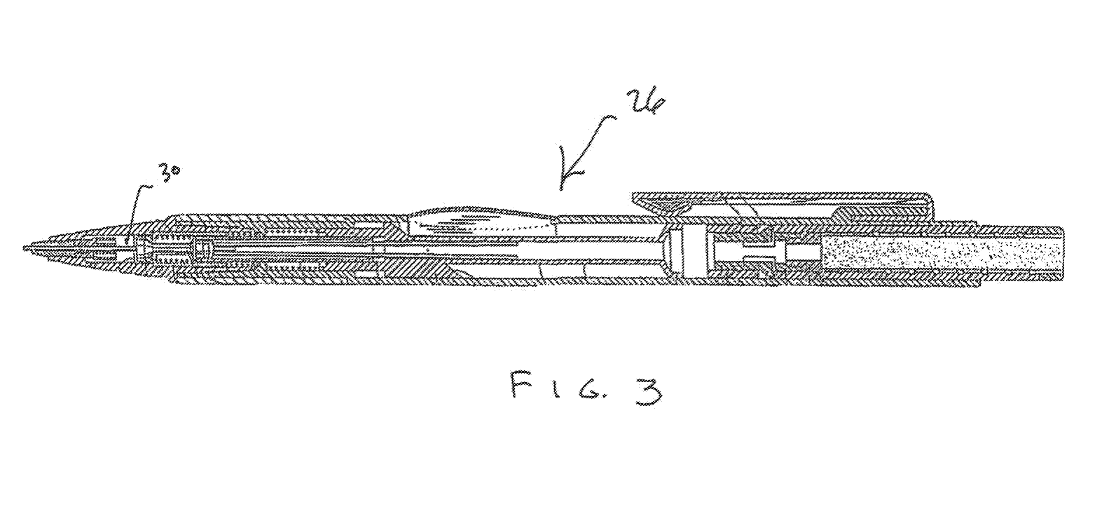

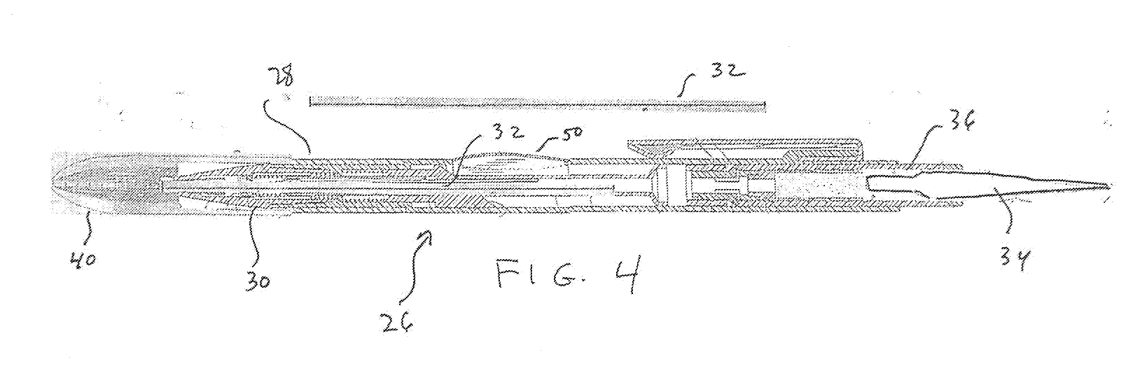

FIG. 3 depicts a conventional mechanical lead pencil 26 as illustrated in U.S. Pat. No. 5,702,193, the entirety of which is hereby incorporated by reference for an illustration as to the manner in which the herein disclosed invention may be practiced. FIG. 4 illustrates an embodiment of the invention utilizing the pencil 26 depicted in FIG. 3. Therein, the mechanical pencil 26, having a synthetic material or plastic barrel 28 has the tip or end 30 threaded as previously described, the lead 30 (FIG. 3) removed and replaced with metal rod 32 (shown in place and external to the mechanical pencil 26 for purposes of illustrations, only).

A dilator 34 of selected shape is shown disposed in the eraser receptacle 36 (once the eraser, not shown) is removed.

Threadably disposed to the tip or end 30 of mechanical pencil 26 is modular, medicament or plug carrying member 40 which, as will be seen, carries discreet medicaments, or in this case punctal plugs, as will become apparent.

Suffice to say that the member 40 is sterilized and medical grade packaged because, in the case or tear duct usage, it is imperative to have and maintain a sterile field.

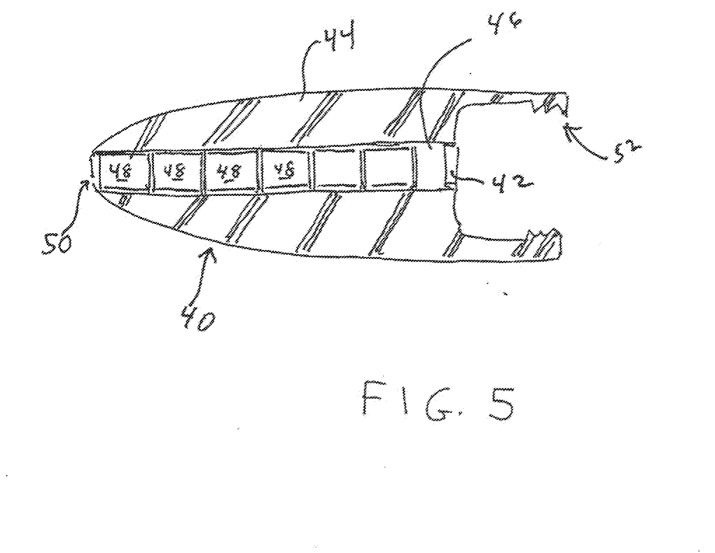

Once the member 40 is operatively engaged, having been taken out of its sterile environment, to mechanical pencil end 30, actuation of side actuator 50 advances metal rod 32 to impact or abut an end member 42 (FIG. 5) to dispel or eject discreet medicaments or plugs, as will be seen, one at a time as selected by physician or other person utilizing the invention.

Referring to FIG. 5 wherein the medicament or plug carrying member 40 is illustrated, it will be noted that member 40 is of plastic or other synthetic material construction. Member 40 has an outer body 44 which has a center bore 46 in which are disposed, in this case, plugs 48, the center bore 46 terminating in opening 50. The size of the plugs 48 are normally 1.2-3.2 mm in length and from 0.2-1.2 mm in width and are frictionally retained in the bore 46 and may be easily moved there along upon axial pressure being applied by the movement of rod 32, actuated by side actuator 50 to impinge upon impenetrable member 42 which acts as a piston to dispel, in serial fashion, the plugs 48 or discreet medicament. In the case of punctal plug usage, the same may be of collagen, synthetic absorbable or silicone types and those of ordinary skill in the art will be familiar with their makeup and prescribed uses.

It will be noted that the orifice end 50 of member 44 is opposite threaded end 52 by which the member 44 is secured to the pencil tip 30.

### EXAMPLE

In the practice of the invention, a patient presents and it is determined that the condition requires the insertion of punctal plugs in each of the tear ducts of the patient.

In preparation for the procedure, an ubiquitous, conventional mechanical lead pencil with a side actuator is prepared by removing the contained lead and replacing it with a provided thin metal rod. The plastic end of the pencil is mechanically threaded by use of a threader. A sterile package carrying a modular member having a predetermined number of and selected punctal plugs, and a dilator is opened.

The patient's head is placed in the slit lamp, with chin on chin rest, and forehead touching forehead rest; patient is prepared for the punctal dilation and/or punctal insertion into the patient's punctum or tear ducts.

The plug carrying member is cooperatively secured to the end of the mechanical pencil (by means of the mating threads). The dilator is inserted into the opposite eraser carrying end of the pencil wherein the eraser has been removed.

The tear duct is dilated by the dilator and within the 4-5 seconds that the tear duct remains open, the plug carrying member end, of the by now unitary device, is placed into the duct opening, the side actuator of the mechanical pencil is actuated to advance the metal rod insert which in turn impinges on the barrier, impenetrable member which may be of plastic, which in turn exerts an axial forward or dispensing force upon the tandem spaced plugs to dispel or dispense one of the plugs into the tear duct or punctum.

The remaining tear ducts and plugs are similarly treated.

While the invention has been described wherein a mechanical lead pencil is utilized, those of ordinary skill in the art will at once recognize that an OEM or original manufactured device is also completed, wherein the entire instrument is fabricated of medical grade disposable plastic or other materials wherein plug dispenser, actuator and dilator are designed and fabricated as a stand-alone device suited for the medical ends as described herein.

Additionally, while the device has been specifically described for use in the ophthalmology area, those of skill in the art will recognize that the unique and novel device of the inventor may be used wherever it is deemed necessary to dispense a medicament in a natural orifice of a body.

The forgoing description of the preferred embodiments of the invention have been presented for the purposes of illustration and description. It is not intended to be exhaustive or to limit the invention to the precise form disclosed. Many modification and variations are possible in light of the above teachings. It is intended that the scope of the invention not be limited by this detailed description, but by the claims and the equivalents to the claims appended hereto.

What is claimed is:

1. A medical device for the placement of medicinal compounds in a natural orifice of a body;
said medical device comprising:
(a) A medicinal compound carrying portion; housing discreet medicaments spaced in tandem relationship.
(b) A terminus of said medical device being adapted for insertion into said natural orifice;
(c) A discharge portion for discharging one of said medical of said medicinal compound into said natural orifice.

2. The medical device in accordance with claim 1 wherein said medical compound carrying portion contains a plurality of discrete medical compounds, frictionally held therein and which may be one at a time serially dispensed.

3. The medical device in accordance with claim 2 wherein said medical compound carrying portion is elongated and is adapted to house said plurality of medical compounds in an end to end manner.

4. The medical device in accordance with claim 3 which additionally includes an actuator for serially dispensing said discreet medicinal compound.

5. The medical device in accordance with claim 4 wherein said body is that of a human and said actuator encompasses an elongated shaft to exert sufficient force upon said medical compound in discreet form to expel same into said natural orifice.

6. A medical device for dispensing punctal plugs into the punctum of a human comprising:
(a) An elongated barrel member for housing a plurality of tandem spaced punctal plugs and having a first end and a second end;
(b) said first end being adapted for ease of insertion into the punctum opening and the second end being adapted to apply dispelling forces to said tandem spaced punctal plugs such that only one of said punctal plugs is dispelled at a time.
(c) whereby upon actuation a punctal plug is ejected into the punctum.

7. A medical device in accordance with claim 6 wherein said second end is configured as a dilator to facilitate opening of said punctum to allow ease of insertion of said first end.

8. A medical device in accordance with claim 7 wherein said first end is modular and is adapted for cooperative attachment to a mechanical lead pencil.

9. A medical device in accordance with claim 8 wherein actuation of said mechanical lead pencil serves as the dispelling force on said punctal plugs.

10. A medical device in accordance with claim 9 wherein said mechanical lead pencil has the lead member removed and replaced with an elongated rod for exerting dispensing force on said punctal plugs.

11. A medical device for serially inserting punctal plugs in selected and predetermined fashion into the punctum of a human comprising:
(a) An elongated tubular member for housing a plurality of tandem spaced punctal
plugs and having a first end configured for ease of insertion into said punctum and to allow passage of said punctal plugs therethrough, said elongated tubular member having a second end having a dispensing means for exerting axial force upon said tandem spaced punctal plugs to dispense one of said puntal plugs at a time through said first end and into said punctual, said elongated tubular member being a mechanical lead pencil with the lead element being removed and having a front end having screw threads and said first end being a modular member having mating screw threads for cooperative attachment to said front end of said mechanical lead pencil, said mechanical lead pencil with the lead removed forming a punctal plug chamber for receiving said punctal plugs in end on end relationship; said mechanical lead pencil with the lead removed also including an elongated rod member adapted to be received within said elongated tubular member with one frontal end therein being adapted to exert axial dispensing force on said punctal plugs wherein only one is dispelled at a time and the opposed distal end being configured as a dilator for dilating the punctum.

12. A medical device in accordance with claim 6 wherein said medical device is fabricated of plastic and said punctal plugs are of a selected size and shape.

13. A medical device in accordance with claim 12 wherein said medical device is fabricated and sterile packaged for one time use.

14. A medical device in accordance with claim 4 wherein said actuator is positioned radially of said medical compound carrying portion and is adapted to exert axial forces on said discrete medical compounds.
